(19) United States
(12) Patent Application Publication
(10) Pub. No.: US 2025/0252565 A1
(43) Pub. Date: Aug. 7, 2025

(54) MEDICAL IMAGE PROCESSING DEVICE AND MEDICAL IMAGE PROCESSING METHOD

(71) Applicant: CANON MEDICAL SYSTEMS CORPORATION, Otawara-shi (JP)

(72) Inventors: Akihiro TAGUCHI, Kawasaki (JP); Ryo SHIROISHI, Kawasaki (JP); Takashi TSUTSUMI, Chigasaki (JP)

(73) Assignee: CANON MEDICAL SYSTEMS CORPORATION, Otawara-shi (JP)

(21) Appl. No.: 19/036,477

(22) Filed: Jan. 24, 2025

(30) Foreign Application Priority Data
Feb. 2, 2024 (JP) 2024-015221

(51) Int. Cl. G06T 7/00; G16H 10/60; G16H 20/40; G16H 30/40

(52) U.S. Cl. CPC G06T 7/0012; G16H 10/60; G16H 20/40; G16H 30/40; G06T 2207/20081; G06T 2207/30101

(57) ABSTRACT

A medical image processing device of an embodiment includes processing circuitry. The processing circuitry acquires a medical image in which at least blood vessels of a patient are captured and patient information regarding the patient, estimates a state of a thrombus captured in the medical image based on the medical image and the patient information, outputs an estimated result related to the estimated thrombus, determines a thrombus removal method for removing the thrombus based on the medical image, the patient information, and the estimation result, outputs a result of determination of the thrombus removal method, generates a display image for presenting any one or a plurality of the medical image, the estimation result, and the determination result, and causes a display device to display the display image.

### CROSS-REFERENCE TO RELATED APPLICATION

The present application claims priority based on Japanese Patent Application No. 2024-015221, filed Feb. 2, 2024, the content of which is incorporated herein by reference.

### FIELD

Embodiments disclosed in this specification and drawings relate to a medical image processing device and a medical image processing method.

### BACKGROUND

For example, in surgery for cerebral infarction, such as ischemic stroke due to occlusion of the main cerebral artery, a thrombus that has occurred inside the blood vessels of a patient, which is the cause of the stroke, is removed. At this time, the surgeon, such as a doctor performing the surgery, determines the location of the thrombus by referring to signs of thrombus captured in medical images, such as CT images captured by a computed tomography (CT) device and MR images captured by a magnetic resonance imaging (MRI) device. However, since the blood vessels and thrombus of the patients are sometimes not clearly visible in CT images or MR images, it is not easy to determine the location of the thrombus.

In relation to this, there have been conventional proposals for information processing devices that estimate an infarcted area and the location of occlusion of a major artery from a CT image, identify the region controlled by occluded blood vessels on the basis of the location of the occlusion of the major artery, and derive and present the amount of overlap (volume) between the controlled region and the infarcted area as a quantitative value. In the conventional technology, a CT image is divided into predetermined regions, such as a region controlled by the left and right anterior cerebral arteries, a region controlled by the middle cerebral artery, and a region controlled by the posterior cerebral artery, and the CT image and the quantitative value are displayed by superimposing the derived quantitative value on each region.

Furthermore, in recent years, technology for collecting stroke thrombi using thrombus removal devices for removing thrombi, such as stent retrievers and suction catheters, has advanced, and detailed analysis of the morphological and histological composition of thrombi can be performed by pathological analysis of thrombi taken (removed) from patients. As a result, it has become clear that thrombi are very heterogeneous in composition and are composed of, for example, fibrin, platelets, red blood cells, white blood cells, von Willebrand Factor (vWF), and neutrophil extracellular traps (NETs). Here, cerebral infarction is broadly classified into cardiogenic cerebral embolism, atherothrombotic brain infarction (ATBI), lacunar infarction, strokes due to other confirmed causes, and strokes due to unconfirmed causes. It has become possible to estimate the constituents of a thrombus taken from a patient and captured in a medical image by identifying the constituents of the thrombus and learning the relationship between feature amounts in the region of the thrombus captured in the medical image and the identified constituents through machine learning or the like. It is conceived that, if the constituents of a thrombus can be estimated, the hardness of the thrombus can be determined and a suitable surgical procedure for removing the thrombus can be determined.

However, in estimation of the constituents of a thrombus captured in a medical image, constituents of the entire thrombus region can be estimated, but it is not yet possible to estimate the local constituents in the thrombus region. For this reason, it is difficult to determine a surgical procedure for removing the thrombus, including the resistance of the thrombus to a thrombus removal device used to remove the thrombus and a thrombus dissolving agent to dissolve the thrombus.

### BRIEF DESCRIPTION OF THE DRAWINGS

FIG. 1 is a diagram showing an example of a functional configuration of a medical image processing device according to an embodiment.

FIG. 2 is a diagram showing an example of thrombus removal methods and segmentation methods determined in advance in the medical image processing device according to the embodiment.

FIG. 3 is a diagram showing an example (part 1) of image processing in which a region segmentation image processing function included in the medical image processing device according to the embodiment segments a thrombus region image.

FIG. 4 is a diagram showing an example (part 2) of image processing in which the region segmentation image processing function included in the medical image processing device according to the embodiment segments a thrombus region image.

FIG. 5 is a diagram showing an example (part 3) of image processing in which the region segmentation image processing function included in the medical image processing device according to the embodiment segments a thrombus region image.

FIG. 6 is a diagram showing an example (part 4) of image processing in which the region segmentation image processing function included in the medical image processing device according to the embodiment segments a thrombus region image.

FIG. 7 is a diagram showing an example (part 5) of image processing in which the region segmentation image processing function included in the medical image processing device according to the embodiment segments a thrombus region image.

FIG. 8 is a diagram showing an example of differences in regions for which feature values are calculated by the region segmentation image processing function included in the medical image processing device according to the embodiment.

FIG. 9 is a diagram showing an example of processing when feature values of segmented regions are calculated by the region segmentation image processing function included in the medical image processing device according to the embodiment.

FIG. 10 is a diagram showing an example (part 1) of a display image generated by a region display function included in the medical image processing device according to the embodiment.

FIG. 11 is a diagram showing an example (part 2) of a display image generated by the region display function included in the medical image processing device according to the embodiment.

FIG. 12 is a diagram showing an example (part 3) of a display image generated by the region display function included in the medical image processing device according to the embodiment.

FIG. 13 is a flowchart showing an example of a processing flow in the medical image processing device according to the embodiment.

FIG. 14 is a diagram showing an example of a display screen when the medical image processing device according to the embodiment provides information.

### DETAILED DESCRIPTION

A medical image processing device of an embodiment includes processing circuitry. The processing circuitry acquires a medical image in which at least blood vessels of a patient are captured and patient information regarding the patient, estimates a state of a thrombus captured in the medical image based on the medical image and the patient information, outputs an estimated result related to the estimated thrombus, determines a thrombus removal method for removing the thrombus based on the medical image, the patient information, and the estimation result, outputs a result of determination of the thrombus removal method, generates a display image for presenting any one or a plurality of the medical image, the estimation result, and the determination result, and causes a display device to display the display image.

A medical image processing device and a medical image processing method of an embodiment will be described below with reference to the drawings.

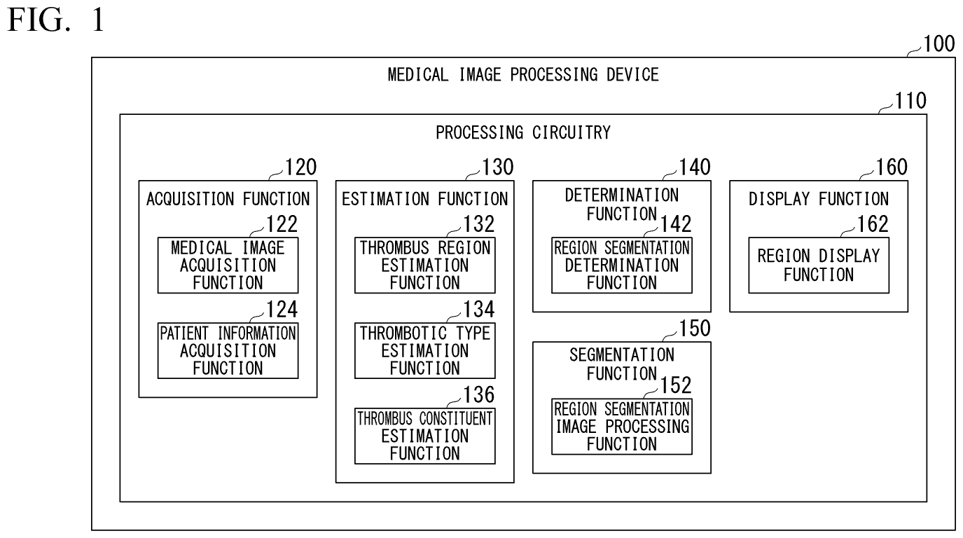

FIG. 1 is a diagram showing an example of a functional configuration of a medical image processing device according to an embodiment. The medical image processing device 100 causes a display device (not shown), such as a liquid crystal display (LCD) for presenting information, to display information regarding a thrombus, thereby presenting the information to a user of the medical image processing device 100, such as a doctor who diagnoses the cerebral infarction (thrombus) or a surgeon who performs surgery to remove the thrombus.

The medical image processing device 100 is realized by a computer device such as a personal computer (PC) installed in, for example, a consultation room or an operating room of a hospital. When the medical image processing device 100 is realized by a personal computer, a display device (not shown) for presenting information to a user is connected to the medical image processing device 100. An input interface for a user to operate the medical image processing device 100 or input information may be connected to the medical image processing device 100. The display device and the input interface may be connected to the medical image processing device 100 through wireless communication. The medical image processing device 100 may be realized by a server device on a network (not shown). In this case, at least a display device (which may include an input interface) may be installed in a consultation room or an operating room, and the server device, which is the main device of the medical image processing device 100, communicates with the display device (which may include an input interface) via a network (not shown). Furthermore, in the medical image processing device 100, only some of the functions which will be described below may be realized by a server device, and in this case, the server device, which is the main device of the medical image processing device 100, and the server device in which some functions are realized communicate with each other via a network (not shown).

The network (not shown) includes, for example, the Internet, a wide area network (WAN), a local area network (LAN), a provider device, a wireless base station, and the like. The input interface is realized by, for example, a mouse, a keyboard, a touch panel, a microphone, and the like. When the input interface is a touch panel, the input interface may be formed integrally with a display device connected to the medical image processing device 100. In this specification, the input interface is not limited to only those having physical operating parts such as the mouse and keyboard described above. For example, examples of the input interface also include an electrical signal processing circuit that receives an electrical signal corresponding to an input operation from an external input apparatus provided separately from the medical image processing device 100 and outputs this electrical signal to the medical image processing device 100.

### Functional Configuration of Medical Image Processing Device

The medical image processing device 100 includes, for example, processing circuitry 110. The processing circuitry 110 executes, for example, processing of an acquisition function 120, an estimation function 130, a determination function 140, a segmentation function 150, a display function 160, and the like. The acquisition function 120 executes, for example, processing of a medical image acquisition function 122, a patient information acquisition function 124, and the like. The estimation function 130 executes, for example, processing of a thrombus region estimation function 132, a thrombotic type estimation function 134, a thrombus constituent estimation function 136, and the like. The determination function 140 executes, for example, processing of a region segmentation determination function 142 and the like. The segmentation function 150 executes, for example, processing of a region segmentation image processing function 152 and the like. The display function 160 executes, for example, processing of a region display function 162 and the like.

The processing circuitry 110 realizes the functions of the acquisition function 120 (including the medical image acquisition function 122 and the patient information acquisition function 124), the estimation function 130 (including the thrombus region estimation function 132, the thrombotic type estimation function 134, and the thrombus constituent estimation function 136), the determination function 140 (including the region segmentation determination function 142), the segmentation function 150 (including the region segmentation image processing function 152), and the display function 160 (including the region display function 162), for example, by a hardware processor executing a program (software) stored in a memory (storage) (not shown). The memory (not shown) is realized by, for example, a semiconductor memory element such as a read only memory (ROM), a random access memory (RAM), or a flash memory, a hard disk drive (HDD), an optical disc, or the like.

For example, the hardware processor means circuitry such as a central processing unit (CPU), a graphics processing unit (GPU), a large scale integration (LSI) circuit, a system on chip (SOC), an application specific integrated circuit (ASIC), a programmable logic device (e.g., a simple programmable logic device (SPLD) or a complex programmable logic device (CPLD), or a field programmable gate array (FPGA)). Instead of storing a program in a memory (not shown), the program may be directly embedded in the circuit of the hardware processor. In this case, the hardware processor realizes each function by reading and executing the program embedded in the circuit. The hardware processor is not limited to being configured as single circuitry, and may be configured as one hardware processor by combining a plurality of independent circuits to realize each function. A plurality of components may be integrated into one hardware processor to realize each function. A plurality of components may be built into one dedicated LSI circuit to realize each function. Here, the program (software) may be stored in advance in a storage device (storage device including a non-transient storage medium) constituting a storage device such as a semiconductor memory element such as a ROM, a RAM, or a flash memory, or a hard disk drive (HDD), or may be stored in a removable storage medium (non-transient storage medium) such as a DVD or a CD-ROM, and may be installed in the storage device of the medical image processing device 100 by setting the storage medium in a drive device of the medical image processing device 100. The program (software) may be downloaded in advance from another computer device via a network (not shown) and installed in the storage device of the medical image processing device 100. The program (software) installed in the storage device of the medical image processing device 100 may be transferred to the processing circuitry included in the medical image processing device 100 and executed.

The acquisition function 120 acquires medical images and patient information. The acquisition function 120 acquires medical images of a patient captured during the current diagnosis or surgery. The acquisition function 120 may acquire, for example, medical images of the patient recorded in a recording device such as picture archiving and communication systems (PACS) that manage data of medical images via a network (not shown). The acquisition function 120 acquires patient information (clinical information other than medical images) regarding the patient undergoing the current diagnosis or surgery. The acquisition function 120 may acquire patient information from a recording device that records data such as examination results from previous examinations of the patient and medical records (electronic medical records) from when the patient was examined. The acquisition function 120 outputs the acquired medical images and patient information to the estimation function 130 and the display function 160.

The medical image acquisition function 122 acquires medical images. The medical images are mainly images of blood vessels in the patient's brain captured at the time of diagnosing a stroke. The medical images include, for example, non-contrast-enhanced CT (NCCT) images (hereinafter, simply referred to as “CT images”) captured by a computed tomography (CT) device, CT angiography (CTA) images, 4-dimensional-CT perfusion (CTP) images, and the like. The medical images also include, for example, MR images captured by a magnetic resonance imaging (MRI) device, MR angiography (MRA) images, MR perfusion (MRP) images, and the like. The medical images also include, for example, angiography images, ultrasound images captured by an ultrasound diagnostic device, single photon emission computed tomography (SPECT) images captured by a SPECT device, and the like. These medical images are merely examples, and the medical images acquired by the medical image acquisition function 122 may include other medical images as long as they are images in which blood vessels (blood flow) in the patient's brain that are necessary for diagnosing a stroke are captured. For example, the medical images may include dual energy CT (DECT) images captured by a DECT device, photon-counting CT (PCCT) images captured by a PCCT device. The medical image acquisition function 122 outputs the acquired medical images to each of the estimation function 130 and the display function 160. The medical image acquisition function 122 may be configured to store the acquired medical images in a storage (not shown) and output a notification indicating the same to each of the estimation function 130 and the display function 160. In this case, for example, the estimation function 130 reads the medical images stored in the storage (not shown), which is equivalent to a configuration in which the medical image acquisition function 122 outputs the acquired medical images to the estimation function 130.

The patient information acquisition function 124 acquires patient information. The patient information includes, for example, information that is generally collected when diagnosing a stroke, such as the patient's age, sex, national institutes of health stroke scale (NIHSS) score, blood pressure, medical history (diabetes, hypertension, dyslipidemia, smoking, stroke, myocardial infarction, atrial fibrillation, anticoagulation, etc.), elapsed time since onset, blood test results (brain natriuretic peptide (BNP) etc.), and genotype by genetic test. Such patient information is merely examples, and the patient information acquired by the patient information acquisition function 124 may include other information on the patient. The patient information acquisition function 124 outputs the acquired patient information to each of the estimation function 130 and the display function 160. The patient information acquisition function 124 may be configured to store the acquired patient information in a storage (not shown) and output a notification indicating the same to each of the estimation function 130 and the display function 160. In this case, for example, the estimation function 130 reads the patient information stored in the storage (not shown), which is equivalent to a configuration in which the patient information acquired by the patient information acquisition function 124 is output to the estimation function 130.

The acquisition function 120 (including the medical image acquisition function 122 and the patient information acquisition function 124) is an example of an “acquirer.”

The estimation function 130 estimates the state of a thrombus captured in a medical image on the basis of the medical image and patient information output by the acquisition function 120, that is, on the basis of the medical image output by the medical image acquisition function 122 and the patient information output by the patient information acquisition function 124. The estimation function 130 estimates the region of the thrombus (hereinafter referred to as a “thrombus region”), the disease type (hereinafter referred to as a “thrombotic type”), and the constituent composition (hereinafter referred to as a “thrombus constituent composition”) from image feature amounts (radiological medical features) of the thrombus captured in the medical image. Examples of image feature amounts include morphological feature amounts such as the size and shape of the thrombus, feature amounts based on a frequency distribution of pixel values in the thrombus region (histogram: minimum value, average value, percentile value, etc.), and feature amounts based on spatial distribution of pixel values in the thrombus region (texture: gray-level co-occurrence matrix (GLCM), gray-level run-length matrix (GLRLM), etc.). The estimation function 130 outputs an estimation result regarding the estimated thrombus.

The thrombus region estimation function 132 estimates a thrombus region that is considered to be a thrombus captured in a medical image. The thrombus region estimation function 132 estimates a thrombus region using a machine learning model generated by a computing device (not shown) or the like using, for example, a machine learning function in artificial intelligence (AI). The machine learning model is a trained model that has been trained in advance to output an image in which a region of hyperdense artery sign (HAS), which indicates a thrombus symptom that can be confirmed on a medical image, has been determined (annotated) as a thrombus region when the medical image is input thereto using a machine learning technique such as a convolutional neural network (CNN), for example. The thrombus region estimation function 132 inputs, for example, an NCCT image (CT image) of the brain of a patient captured before thrombus removal to the machine learning model, thereby obtaining an image in which a region of HAS on the NCCT image has been divided (segmented) as a thrombus region. This image is associated with information indicating the position of the thrombus region on the NCCT image. The medical image from which the thrombus region estimation function 132 estimates the thrombus region is not limited to an NCCT image, and may be any medical image, including the various medical images described above, as long as it shows a thrombus sign that can be assumed to be a thrombus region. The thrombus region estimation function 132 outputs the image (the image of the segmented thrombus region) obtained by the machine learning model as a thrombus region estimation result to each of the determination function 140, the segmentation function 150, and the display function 160. In the following description, the image of the thrombus region estimation result output by the thrombus region estimation function 132 is referred to as a “thrombus region image.”

The thrombotic type estimation function 134 estimates a thrombotic type of a thrombus that is considered to be captured in a medical image on the basis of the medical image and patient information. The thrombotic type estimation function 134 may use a thrombus region image in which a thrombus region has been estimated by the thrombus region estimation function 132 at the time of estimating a thrombotic type. The thrombotic type indicates a cause of a disease such as cardiogenic cerebral embolism, atherothrombotic brain infarction (ATBI), lacunar infarction, and embolic stroke of undetermined source (ESUS). The thrombotic type estimation function 134 estimates a thrombotic type using a machine learning model generated by a computing device (not shown) or the like using a machine learning function in AI, for example. The machine learning model is a trained model that has been trained in advance to output a thrombotic type identified after thrombus collection in a cerebral infarction patient when image feature amounts of a medical image of the cerebral infarction patient captured before thrombus removal and patient information are input thereto using a machine learning technique such as a support vector machine (SVM). The thrombotic type estimation function 134 obtains information on the thrombotic type estimated before thrombus collection, for example, by inputting image feature amounts of a medical image of the patient and the patient information to the machine learning model. The information on the thrombotic type is information that indicates the probability of each thrombotic type, such as “atheromatous=0.8%” and “cardiogenic=0.2%.” The information on the thrombotic type may be, for example, binary information indicating whether the thrombotic type is atheromatous or cardiogenic, by determining the probability of each thrombotic type using a predetermined threshold value. Such information on the thrombotic type is merely examples, and the information on the thrombotic type obtained by estimating the thrombotic type by the thrombotic type estimation function 134 may be any information on the thrombotic type. The thrombotic type estimation function 134 outputs the information on the thrombotic type obtained by the machine learning model as a thrombotic type estimation result to each of the determination function 140, the segmentation function 150, and the display function 160. In the following description, the information on the thrombotic type estimation result output by the thrombotic type estimation function 134 is referred to as “thrombotic type information.”

The thrombus constituent estimation function 136 estimates the thrombus constituent composition of a thrombus that is considered to be captured in a medical image on the basis of the medical image and patient information. When a patient has a plurality of thrombi, such as when the patient has a tandem occlusion in the cerebral infarction, the thrombus constituent estimation function 136 may estimate the thrombus constituent composition of each thrombus that has occurred in the patient. The thrombus constituent composition represents, for example, a constituent composition of fibrin, platelets, red blood cells, white blood cells, and the like. The thrombus constituent estimation function 136 estimates, for example, the number of red blood cells (red blood cell: RBC) as the thrombus constituent composition. The thrombus constituent estimation function 136 estimates the thrombus constituent composition using, for example, a machine learning function in AI, using a machine learning model generated by a computing device (not shown) or the like. The machine learning model is a trained model that has been trained in advance to output results of analysis of histopathological diagnosis after thrombus collection in a cerebral infarction patient when image feature amounts of a medical image of the cerebral infarction patient captured before the thrombus is removed and patient information are input thereto using a machine learning technique such as a CNN or a support vector machine. In the histopathological diagnosis, for example, the proportion of each constituent constituting the thrombus is analyzed by staining (e.g., H&S staining) a section of the thrombus removed and collected from the cerebral infarction patient and analyzing the same using an optical microscope. The analysis results are represented, for example, as “RBC=aa %, fibrin/platelet=bb %, white blood cell=cc %” with the total being 100%. For example, the thrombus constituent estimation function 136 obtains information on the thrombus constituent composition estimated before thrombus collection by inputting image feature amounts of the medical image of the patient and the patient information to the machine learning model. The thrombus constituent estimation function 136 obtains, for example, information which indicates the proportion of each component constituting the thrombus, such as “RBC=0.8%, fibrin/platelet=0.2%,” as information on the thrombus constituent composition. For example, the hardness of the thrombus can be determined based on the proportion of RBC and fibrin/platelet in the thrombus. More specifically, it can be determined that the lower the proportion of RBC (higher fibrin/platelet), the harder the thrombus is, and the higher the proportion of RBC (lower fibrin/platelet), the softer the thrombus is. Such information on the thrombus constituent composition is merely an example, and the information on the thrombus constituent composition obtained by the thrombus constituent estimation function 136 estimating the thrombus constituent composition may be any information as long as it is information on the thrombus constituent composition. The thrombus constituent estimation function 136 outputs the information on the thrombus constituent composition obtained by the machine learning model to each of the determination function 140, the segmentation function 150, and the display function 160 as a result of estimation of the thrombus constituent composition. In the following description, information on the result of estimation of the thrombus constituent composition output by the thrombus constituent estimation function 136 is referred to as “thrombus constituent composition information.”

The estimation function 130 is an example of an “estimator.” The thrombus region estimation function 132 is an example of a “thrombus region estimator.” The thrombotic type estimation function 134 is an example of a “thrombotic type estimator.” The thrombus constituent estimation function 136 is an example of a “thrombus constituent estimator.”

The determination function 140 determines a method of removing the thrombus captured in the medical image (hereinafter referred to as a “thrombus removal method”) on the basis of the images and information of the respective estimation results output by the estimation function 130. More specifically, the determination function 140 determines the thrombus removal method on the basis of the thrombus region image output by the thrombus region estimation function 132, the thrombotic type information output by the thrombotic type estimation function 134, and the thrombus constituent composition information output by the thrombus constituent estimation function 136.

The region segmentation determination function 142 determines a method of segmenting the thrombus region captured in the medical image, i.e., the thrombus region represented by the thrombus region image, on the basis of a combination of the thrombus region image, thrombotic type information, and thrombus constituent composition information, and the determination result of the determination function 140 on the thrombus removal method. More specifically, the region segmentation determination function 142 determines which segmentation method which will be described will be used by the segmentation function 150 to segment the thrombus region.

The thrombus removal method determined by the determination function 140 and the segmentation method determined by the region segmentation determination function 142 may be set, for example, by a user performing an input operation on an input interface (not shown), or may be determined in advance for each combination of estimated results of the thrombus region image, thrombotic type information, and thrombus constituent composition information.

### Example of Thrombus Removal Method and Segmentation Method

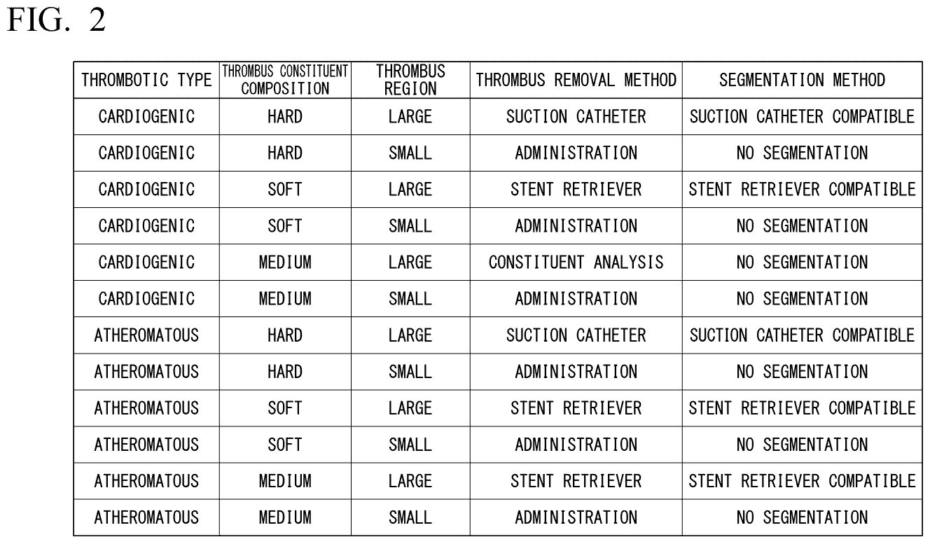

FIG. 2 is a diagram showing an example of thrombus removal methods and segmentation methods determined in advance in the medical image processing device 100 according to the embodiment. FIG. 2 shows an example of a case in which and the hardness of a thrombus is divided into three types, “hard,” “soft,” and “medium,” depending on the proportion of fibrin in a thrombus constituent composition represented by thrombus constituent composition information, and the size of the thrombus is divided into two types, “large” and “small,” depending on the volume, diameter, and the like of a thrombus region represented by a thrombus region image, for a case in which a thrombotic type represented by thrombotic type information is “atheromatous” and “cardiogenic.” The number of types into which the hardness and size of the thrombus shown in FIG. 2 are divided is merely an example, and the thrombus may be divided into more types. That is, the hardness and size of the thrombus may be divided into more fine categories. The hardness of the thrombus can be divided on the basis of a threshold value set for the proportion of fibrin represented by thrombus constituent composition information, for example. The size of the thrombus can be divided on the basis of a threshold value set for the size of a thrombus region represented by thrombus region image, for example.

In the example shown in FIG. 2, when the hardness of the thrombus is “hard” and the size of the thrombus is “large” regardless of the thrombotic type, a procedure using a suction catheter as a thrombus removal device is set as a thrombus removal method, and a segmentation method corresponding to the suction catheter is set. This is because, in the case of a hard thrombus, it is generally considered that if a stent retriever is used as a thrombus removal device, the stent retriever inserted into the thrombus is not sufficiently expanded, and there is a high possibility that a part of the thrombus cannot be removed, and thus it is more preferable to use a suction catheter. In the example shown in FIG. 2, when the hardness of the thrombus is “soft” and the size of the thrombus is “large” regardless of the thrombotic type, and in addition, when the thrombotic type is “atheromatous,” the hardness of the thrombus is “medium,” and the size of the thrombus is “large,” a procedure using a stent retriever as a thrombus removal device is set as a thrombus removal method, and a segmentation method corresponding to the stent retriever is set. This is because, in the case of a soft thrombus having a certain size or more, it is considered that the stent retriever inserted into the thrombus is sufficiently expanded and the thrombus can be removed. In the example shown in FIG. 2, when the size of the thrombus is “small” regardless of the thrombotic type and the hardness of the thrombus, the thrombus removal method is set to “administration” and the segmentation method is set to “no segmentation” of the thrombus region. This is because it is considered that a small thrombus may be dissolved (removed) by administering a thrombolytic agent for dissolving the thrombus, such as intravenous alteplase (tPA) therapy. In this case, the user may check the overall state of the thrombus from estimated results of a thrombus region image, thrombotic type information, and thrombus constituent composition information and determine a surgical procedure for removing the thrombus. In the example shown in FIG. 2, when the thrombotic type is “cardiogenic,” the hardness of the thrombus is “medium,” and the size of the thrombus is “large,” the thrombus removal method is set to “constituent analysis” and the segmentation method is set to “no segmentation.” That is, in this case, constituent analysis is recommended as a thrombus removal method. This is because a thrombus in this state may have extremely hard or soft local regions, and thus it is considered that it is desirable to calculate a constituent distribution first to ascertain the distribution of thrombus constituent composition before determining a surgical procedure.

In this way, the determination function 140 determines a thrombus removal method on the basis of a combination of a thrombus region image, thrombotic type information, and thrombus constituent composition information, and the region segmentation determination function 142 further determines a segmentation method for the segmentation function 150 to segment a thrombus region in addition to the thrombus removal method. The determination function 140 outputs information representing the determined thrombus removal method (hereinafter simply referred to as a “thrombus removal method”) to the segmentation function 150 and the display function 160. The thrombus removal method includes information on “thrombotic type,” “thrombus constituent composition,” and “thrombus region” shown in FIG. 2. In addition to the information shown in FIG. 2, these pieces of information include values represented by the corresponding information output by the estimation function 130 (such as the probability of the disease type of the thrombus and the proportion of each constituent constituting the thrombus). The region segmentation determination function 142 outputs information representing the determined segmentation method (hereinafter simply referred to as a “segmentation method”) to the segmentation function 150 and the display function 160.

Although the determination function 140 determines (selects) one thrombus removal method, and the region segmentation determination function 142 determines (selects) one segmentation method for the segmentation function 150 to segment a thrombus region in the above-described example, the thrombus removal method selected by the determination function 140 and the segmentation method selected by the region segmentation determination function 142 are not limited to one. For example, the determination function 140 may select a plurality of thrombus removal methods and present the same to the user such that the user can compare the presented thrombus removal methods. For example, the region segmentation determination function 142 may select a plurality of segmentation methods and present the same to the user such that the user can compare the presented segmentation methods. For example, the determination function 140 and the region segmentation determination function 142 may select a thrombus removal method and a segmentation method input by the user performing an input operation on an input interface (not shown), that is, the user may manually select the methods.

The determination function 140 is an example of a “determiner.” The region segmentation determination function 142 is an example of an “region segmentation determiner.”

The segmentation function 150 segments a thrombus region image (an image of a divided thrombus region) output by the estimation function 130 (more specifically, the thrombus region estimation function 132) according to the thrombus removal method output by the determination function 140 and the segmentation method output by the region segmentation determination function 142.

The region segmentation image processing function 152 performs image processing for dividing the thrombus region image according to the thrombus removal method and the segmentation method. The region segmentation image processing function 152 may be configured to select a result of segmentation of the thrombus region image according to the thrombus removal method and the segmentation method after segmentation using all segmentation methods. In this case, the region segmentation image processing function 152 can perform image processing for segmenting the thrombus region image in parallel with processing of the region segmentation determination function 142 determining a segmentation method.

### Example of Segmentation of Thrombus Region Image

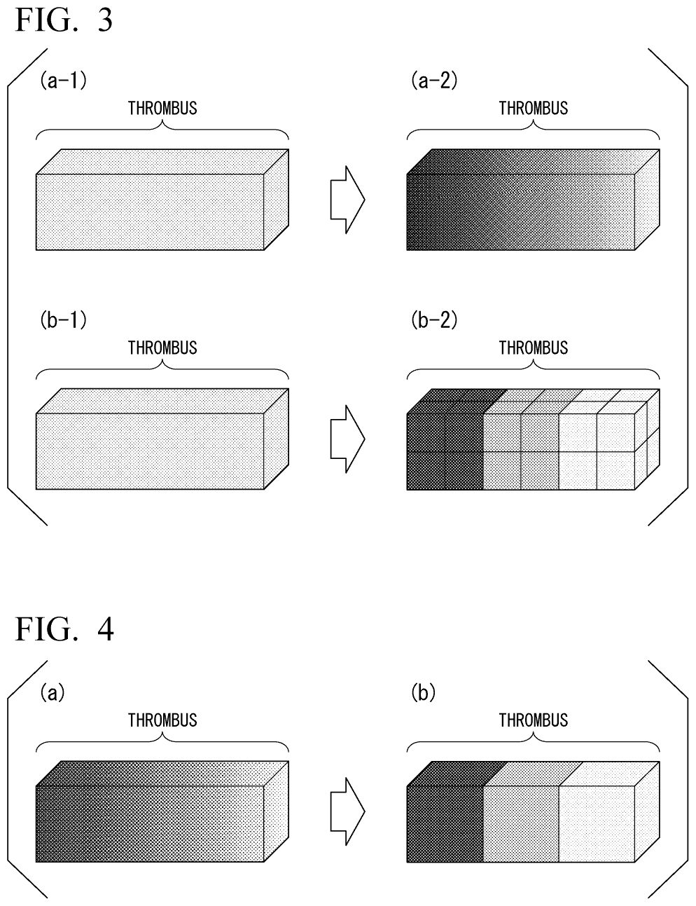

Hereinafter, an example of image processing through which the region segmentation image processing function 152 segments a thrombus region image will be described. FIG. 3 to FIG. 7 are diagrams showing an example of image processing through which the region segmentation image processing function 152 included in the medical image processing device 100 according to the embodiment segments a thrombus region image.

FIG. 3 and FIG. 4 schematically show an example of image processing when a segmentation method indicating “no segmentation” is output from the region segmentation determination function 142. FIG. 3 and FIG. 4 show an example of an image (thrombus region image) of only a thrombus captured in a medical image, estimated by the thrombus region estimation function 132. When a segmentation method indicating “no segmentation” is output from the region segmentation determination function 142, the region segmentation image processing function 152 performs image processing for representing the distribution (constituent distribution) of the thrombus constituent composition estimated by the thrombus constituent estimation function 136 in the entire thrombus (entire thrombus region). At this time, the region segmentation image processing function 152 associates information represented by the thrombus constituent composition as a feature value for each pixel or for each voxel of a predetermined size or a size designated by the user (for example, a cubic voxel of 3 pixels×3 pixels×3 pixels) for respective pixels constituting the thrombus region.

(a) of FIG. 3 schematically shows an example of a case in which information represented by the thrombus constituent composition is associated with each pixel. More specifically, (a-2) of FIG. 3 shows an example of a case in which a distribution of feature values is represented by the brightness (or color) of each pixel for the entire thrombus shown in (a-1) of FIG. 3. The feature value is, for example, a value representing the hardness of the thrombus based on the proportion of RBC and fibrin/platelet represented by the thrombus constituent composition. In this case, the user can check the distribution of feature values of the entire thrombus by gradual changes in contrast (brightness) and color tone (so-called gradation). (b) of FIG. 3 schematically shows an example of a case in which information represented by the thrombus constituent composition is associated with each voxel. More specifically, (b-2) of FIG. 3 shows an example of a case in which the entire thrombus shown in (b-1) of FIG. 3, which is the same as (a-1) of FIG. 3, is segmented into 6 voxels in the horizontal direction, 2 voxels in the vertical direction, and 2 voxels in the depth direction, and the distribution of feature values for each voxel is represented by the brightness (or color) of the voxel. In this case, the region segmentation image processing function 152 may represent the feature value corresponding to the pixel at the center position included in the same voxel as a feature value of the entire voxel as brightness or color, or may average the feature values corresponding to respective pixels included in the same voxel and represent the average value as a feature value of the entire voxel as brightness or color. In this case, the user can check the outline of the distribution of the feature values of the entire thrombus by the contrast (brightness) and color tone of the distribution of feature values. When representing the outline of the distribution of feature values in the thrombus, the region segmentation image processing function 152 may perform image processing as shown in (a-2) of FIG. 3 or (b-2) of FIG. 3 and then perform further image processing to display the outline.

(a) of FIG. 4 schematically shows an example of a case in which image processing for representing the distribution of the feature values of the thrombus shown in (a-2) of FIG. 3 is performed and then image processing for representing the outline of the distribution of the feature values of the thrombus is performed. More specifically, (b) of FIG. 4 shows an example of a case in which the entire thrombus shown in (a) of FIG. 4, which represents the same distribution of feature values as (a-2) of FIG. 3, is segmented into three regions (may be segmented into three voxels) in the horizontal direction, and the distribution of feature values for each region is represented by the brightness or color of the region. In this case, the region segmentation image processing function 152 may represent the feature value corresponding to the pixel at the center position included in the same region or the average feature value as a feature value of the entire region by the brightness or color, as in the case of the voxel shown in (b-2) of FIG. 3. Alternatively, the region segmentation image processing function 152 may, for example, distinguish each region by a threshold value set for the feature value, and represent the entire region by a brightness or color indicating whether the feature value is equal to or greater than the threshold value or equal to or less than the threshold value.

As shown in (b) of FIG. 4, when representing an outline of the distribution of feature values in the thrombus, the region segmentation image processing function 152 may first segment the entire thrombus into suitable sizes (into three in the case of (b) of FIG. 4), and then perform image processing such that each region represents an outline of the distribution of the feature values of the thrombus.

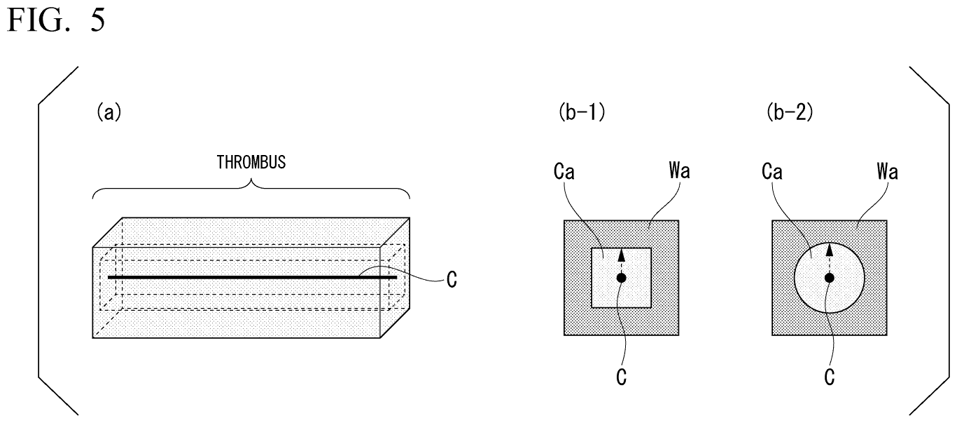

FIG. 5 schematically shows an example of image processing when the determination function 140 outputs a thrombus removal method indicating that a thrombus is “atheromatous.” As in FIG. 3 and FIG. 4, FIG. 5 also shows an example of an image of only the thrombus (thrombus region image) captured in a medical image, estimated by the thrombus region estimation function 132. When the thrombus removal method indicating “atheromatous” is output from the determination function 140, the region segmentation image processing function 152 segments the entire thrombus into two regions, an outer region (blood vessel wall side) and an inner region (center side), performs image processing for each region to represent the distribution of the thrombus constituent composition (constituent distribution) estimated by the thrombus constituent estimation function 136, and associates information represented by the thrombus constituent composition. This is because, in the case of atherothrombotic cerebral infarction, a thrombus may form near a stenosed part of the blood vessel, and the constituents may change in the outer region of the thrombus (region close to the stenosed part) and the inner region (region at the center).

(a) of FIG. 5 shows an example of a case in which the entire thrombus is segmented into an inner region Ca around the central axis C of the thrombus (blood vessel) and an outer region Wa around the inner region Ca, that is, close to the blood vessel wall (stenosed part), and the distribution of feature values for each region is represented by the brightness and color of the region. (b-1) of FIGS. 5 and (b-2) of FIG. 5 show an example of the relationship between the inner region Ca and the outer region Wa when the blood vessel (thrombus) is viewed from the side of the central axis C. (b-1) of FIG. 5 shows an example of a case in which the inner region Ca is represented by a square, assuming that the cross section of the thrombus is square, and (b-2) of FIG. 5 shows an example of a case in which the inner region Ca is represented by a circle in the same manner, assuming that the cross section of the thrombus is square. The difference between (b-1) of FIGS. 5 and (b-2) of FIG. 5 is only the shape of the inner region Ca. The brightness and color of the distribution of feature values shown for each region are representative of the feature values of the same region, as in the examples shown in FIG. 3 and FIG. 4. In such a case, the user can check the difference in feature values between the inside and outside of the thrombus by the contrast (brightness) and color tone of the distribution of feature values.

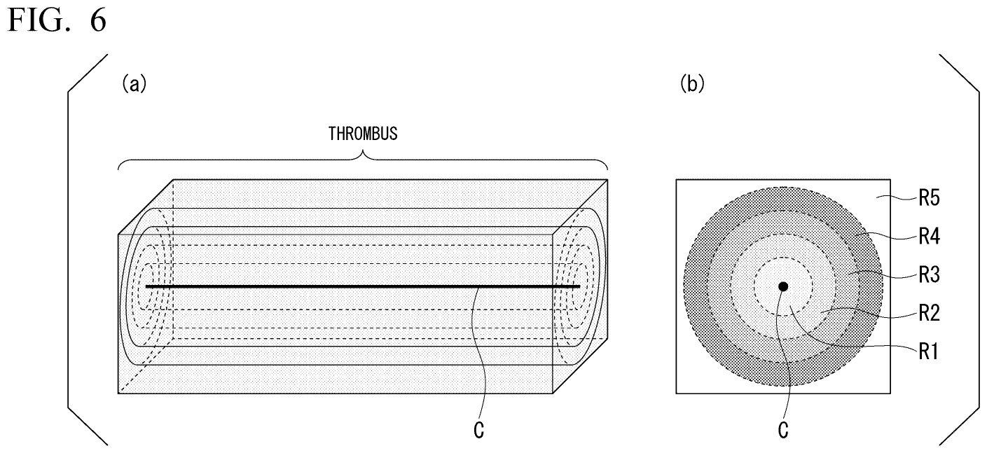

FIG. 6 schematically shows an example of image processing when a segmentation method indicating “stent retriever compatible” is output from the region segmentation determination function 142. As in FIG. 3 to FIG. 5, FIG. 6 also shows an example of an image of only a thrombus (thrombus region image) captured in a medical image, estimated by the thrombus region estimation function 132. When a segmentation method indicating “stent retriever compatible” is output from the region segmentation determination function 142, the region segmentation image processing function 152 segments the entire thrombus into a plurality of regions representing the components of a stent retriever, which is a thrombus removal device, the shape, and the range of deformation of these components when removing the thrombus. This is because when a thrombus is removed using a stent retriever, various components such as a guidewire and a microcatheter are pierced into the thrombus together with the stent retriever, and the thrombus is removed by expansion of the stent retriever. In addition, this is because the type and shape (diameter) of each component, the amount of force required to perforate the thrombus, and the like may vary depending on the shape and hardness (constituent) of the thrombus. The region segmentation image processing function 152 performs image processing for each region to represent the distribution of the thrombus constituent composition (constituent distribution) estimated by the thrombus constituent estimation function 136, and associates information represented by the thrombus constituent composition.

(a) of FIG. 6 shows an example of a case in which the entire thrombus is segmented into, for example, five regions R1 to R5 from the central axis C of the thrombus (blood vessel) toward the periphery, and the distribution of feature values for each region R is represented by the brightness and color of the region. The region R1 is a region that represents the shape (diameter) of the guidewire. The region R2 is a region that represents the shape (diameter) of the stent retriever before expansion. The region R3 is a region that represents the shape (diameter) of the microcatheter in which the guidewire and the stent retriever are housed. The region R4 is a region that represents the shape (maximum diameter) of the stent retriever after expansion. The region R5 is the remaining region, in other words, a region of the thrombus that cannot be removed by the stent retriever. When segmenting the entire thrombus, the region segmentation image processing function 152 calculates, for example, the position of the central axis C of the thrombus region. Then, the region segmentation image processing function 152 creates, for example, cylindrical regions in the normal direction from the calculated central axis C using information on the size of each component (for example, stent diameter, inner diameter, or the like) and sets the cylindrical regions as respective regions R. (b) of FIG. 6 shows an example of the relationship between the regions R1 to R5 when the blood vessel (thrombus) is viewed from the side of the central axis C. The cylindrical regions R may be created in accordance with the size of each component, using the lower base or upper base of the thrombus as a reference instead of the central axis C of the thrombus. The brightness and color of the distribution of feature values shown for each region represent the same feature value, as in the examples shown in FIG. 3 to FIG. 5. In this case, the user can check the difference in the feature values of each region R in the thrombus in a state in which the stent retriever has been perforated, by the contrast (brightness) and color tone of the distribution of feature values.

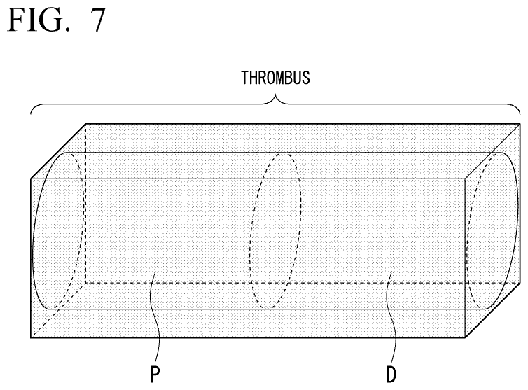

FIG. 7 schematically shows an example of image processing when a segmentation method indicating “suction catheter compatible” is output from the region segmentation determination function 142. As in FIG. 3 to FIG. 6, FIG. 7 also shows an example of an image of only a thrombus (thrombus region image) captured in a medical image, estimated by the thrombus region estimation function 132. When a segmentation method indicating “suction catheter compatible” is output from the region segmentation determination function 142, the region segmentation image processing function 152 segments the entire thrombus from the central axis C of the thrombus (blood vessel) toward the periphery into a plurality of regions according to the components, shape, and size of each component of a suction catheter, which is a thrombus removal device, as in the example shown in FIG. 6, and then segments the same into two regions, a proximal side and a distal side of the thrombus. This is because when a thrombus is removed using a suction catheter, the distal side of the thrombus may be crushed during suction. Furthermore, when a thrombus is removed using a suction catheter, a separator for crushing the thrombus may be used together with the suction catheter, and when the separator crushes the thrombus, the distal side of the thrombus may be crushed and scattered to a further location. In other words, the thrombus that has been crushed and scattered to a further location may become a factor in generation of a new thrombus (i.e., a secondary thrombus). The region segmentation image processing function 152 may further segment the area between the proximal side and the distal side of the thrombus into a plurality of regions according to the distance the suction catheter is advanced when removing the thrombus. The region segmentation image processing function 152 performs image processing for each region to represent the distribution of the thrombus constituent composition (constituent distribution) estimated by the thrombus constituent estimation function 136, and associates information represented by the thrombus constituent composition.

FIG. 7 shows an example of a case in which the entire thrombus is segmented into two regions, a proximal region P and a distal region D, and the distribution of feature values for each region R is represented by the brightness and color of the region. In FIG. 7, the regions segmented from the central axis C toward the periphery are omitted, as in the example shown in FIG. 6. The brightness and color of the distribution of feature values shown for each region represent the feature values of the same region, as in the examples shown in FIG. 3 to FIG. 6. In this case, the user can check the difference in the feature values of each region in the thrombus sucked by the suction catheter by the contrast (brightness) and color tone of the distribution of feature values.

### Example of Method of Calculating Feature Values

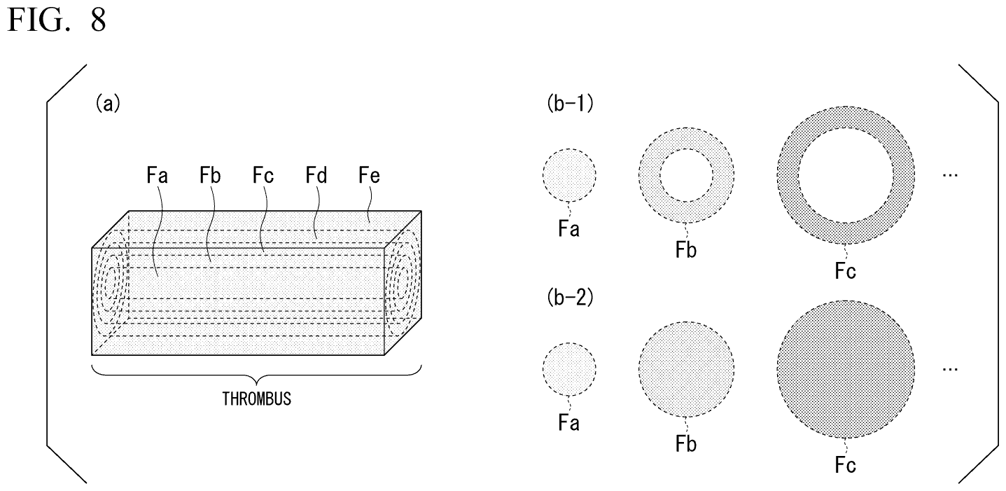

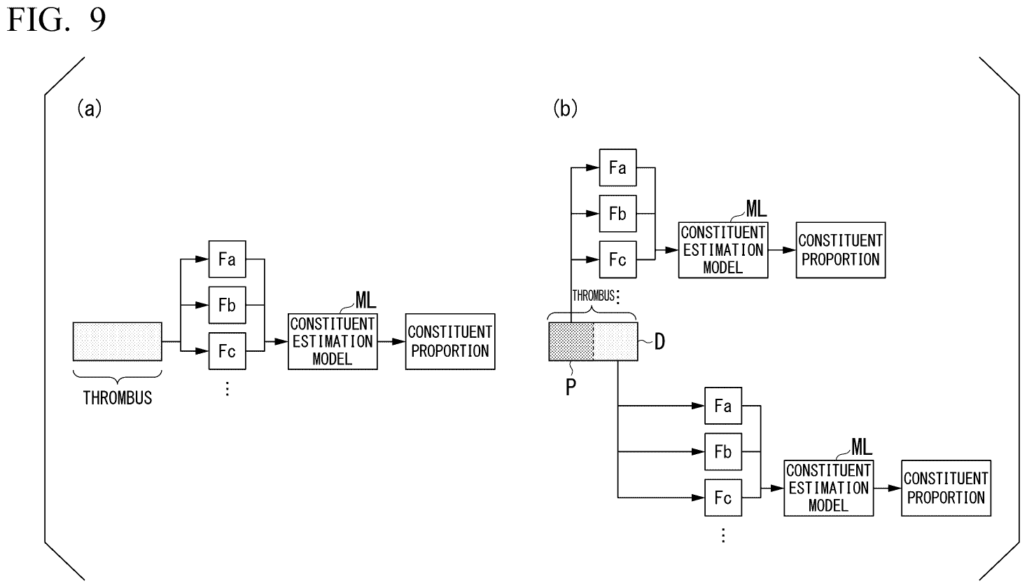

In the examples shown in FIG. 3 to FIG. 7, the feature value corresponding to the pixel at the center position of each segmented region or the average feature value represent the feature values of the same region, but the method of calculating the feature value representing regions is not limited to the above-described examples. An example of a method of calculating the feature value representing segmented regions by the region segmentation image processing function 152 will be described below. FIG. 8 is a diagram showing an example of differences in regions for which feature values are calculated by the region segmentation image processing function 152 included in the medical image processing device 100 according to the embodiment. FIG. 9 is a diagram showing an example of processing when the region segmentation image processing function 152 included in the medical image processing device 100 according to the embodiment calculates feature values of segmented regions.

When performing image processing to represent the distribution of thrombus constituent composition (constituent distribution) estimated by the thrombus constituent estimation function 136, the region segmentation image processing function 152 calculates a feature value of the thrombus constituent composition represented by the thrombus constituent composition information output by the thrombus constituent estimation function 136 for each segmented region. Here, the region segmentation image processing function 152 may calculate feature values of segmented regions only from feature values corresponding to pixels included in the difference regions of the segmented regions, or may calculate the feature values of the segmented regions from feature values corresponding to pixels including an overlapping range in the segmented regions.

FIG. 8 schematically shows an example of differences in the range (region) of feature values used when calculating the feature values. (a) of FIG. 8 shows an example of a case in which the thrombus (blood vessel) is segmented into five regions Fa to Fe that spread from the central axis of the thrombus (blood vessel) toward the periphery, as in the example shown in FIG. 6. (b-1) of FIG. 8 shows an example of a case in which a feature value of a segmented region F is calculated from a feature value of a difference region of the region F. More specifically, the feature value of the region Fa in the center of the thrombus region is calculated using all feature values included in the range of the region Fa. The feature value of the region Fb outside the region Fa is calculated using feature values included in the range obtained by excluding the range of the region Fa from the entire range of the region Fb. Similarly, the feature value of the region Fc outside the region Fb is calculated using feature values included in the range obtained by excluding the range of the region Fb from the entire range of the region Fc. The feature values of the regions Fd and Fe are calculated in a same manner. (b-2) of FIG. 8 shows an example of a case in which the feature value of a region F is calculated including an overlapping range in the segmented region F. More specifically, the feature value of the region Fa is calculated using all the feature values included in the range of the region Fa. The feature value of the region Fb is calculated using all the feature values included in the range of the region Fb, that is, also including the range of the region Fa. Similarly, the feature value of the region Fc is calculated using all the feature values included in the range of the region Fc, that is, also including the ranges of the regions Fa and Fb. The feature values of regions Fd and Fe are calculated in a similar manner.

Then, the region segmentation image processing function 152 inputs the calculated feature values, for example, to a constituent estimation model to calculate a feature value representative of each of the segmented regions. For example, the constituent estimation model is a trained model that has been trained in advance by a computing device (not shown) or the like to output, when calculated feature values are input thereto, the constituent composition of a thrombus, such as the proportion (hereinafter referred to as a “constituent proportion”) of each constituent constituting the thrombus represented by the feature values.

FIG. 9 schematically shows an example of processing of inputting a features value of each region F shown in FIG. 8 to a constituent estimation model ML and calculating (obtaining) a constituent proportion represented by the input feature value. (a) of FIG. 9 schematically shows an example of processing of inputting the features value of each region F shown in FIG. 8 to the constituent estimation model ML and calculating a corresponding constituent proportion. (b) of FIG. 9 schematically shows an example of a case in which the entire thrombus is segmented into two regions, a proximal region P and a distal region D, as in the example shown in FIG. 7, for each region F shown in FIG. 8. The example shown in (b) of FIG. 9 shows an example of processing of inputting a feature value of each region F belonging to the proximal region P to the constituent estimation model ML to calculate a constituent proportion corresponding to the proximal region P, and inputting a feature value of each region F belonging to the distal region D to the constituent estimation model ML to calculate a constituent proportion corresponding to the distal region D.

In this way, the region segmentation image processing function 152 calculates a constituent proportion corresponding to the feature value of each segmented region. Then, as described above, the region segmentation image processing function 152 represents the calculated constituent proportion as the feature value corresponding to each region. The region segmentation image processing function 152 outputs information representing the feature value of each region into which the thrombus region image has been segmented (hereinafter referred to as “region segmentation information”) to the display function 160.

The segmentation function 150 (including the region segmentation image processing function 152) is an example of a “segmenter.”

Referring back to FIG. 1, the display function 160 presents information output by each of the components of the medical image processing device 100 to the user. More specifically, the display function 160 presents, to the user, medical images and patient information output by the acquisition function 120, information on estimation results output by the estimation function 130, a thrombus removal method and a segmentation method output by the determination function 140, and information on regions segmented by the segmentation function 150.

The region display function 162 generates a display image including information to be presented to the user, and presents the generated display image to the user of the medical image processing device 100 by causing a display device (not shown) to output and display the display image. More specifically, the region display function 162 generates a display image for displaying a medical image output by the medical image acquisition function 122, a display image for displaying patient information output by the patient information acquisition function 124, a display image for displaying a thrombus region image output by the thrombus region estimation function 132, a display image for displaying thrombotic type information output by the thrombotic type estimation function 134, a display image for displaying a thrombus constituent composition information output by the thrombus constituent estimation function 136, a display image for displaying a thrombus removal method output by the determination function 140, a display image for displaying a segmentation method output by the region segmentation determination function 142, and a display image for displaying region segmentation information output by the region segmentation image processing function 152. The region display function 162 may generate a single display image by combining some or all of the generated display images (combining or superimposing the display images).

### Example of Display Image

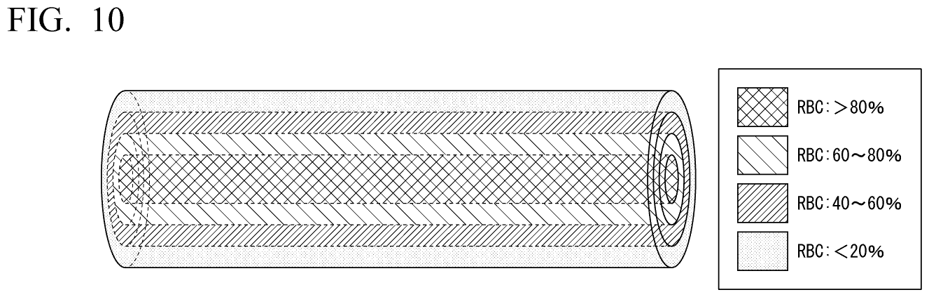

Hereinafter, an example of a display image generated by the region display function 162 will be described. FIG. 10 to FIG. 12 are diagrams showing an example of a display image generated by the region display function 162 included in the medical image processing device 100 according to the embodiment.

FIG. 10 shows an example of a display image for the region segmentation image processing function 152 to display region segmentation information corresponding to regions into which a thrombus region image estimated by the thrombus region estimation function 132 has been segmented. In other words, FIG. 10 shows an example of a display image for presenting, to the user, a distribution (constituent distribution) of a thrombus constituent composition estimated by the thrombus constituent estimation function 136. More specifically, FIG. 10 shows an example of a display image in which, in the case of segmenting a thrombus (blood vessel) into four regions spreading from the central axis toward the periphery, the hardness of each region is presented to the user by a difference in color (hatching in FIG. 10), as in the example shown in FIG. 6. As described above, the hardness of a thrombus can be determined by the proportions of RBC and fibrin/platelets. For this reason, in the example shown in FIG. 10, the proportion of red blood cells (RBC) is displayed to the user to indicate the hardness of each region. More specifically, if the sum of the proportions of RBC and fibrin/platelets is 100%, the proportion of red blood cells in the central region is “RBC: >80%,” that is, “fibrin/platelets<20%,” which indicates that the central region is a softest region. On the other hand, the proportion of red blood cells in the outermost region is “RBC: <20%,” that is, “fibrin/platelets >80%,” which indicates that the outermost region is a hardest region.

When a display image as shown in FIG. 10 is generated, for example, the region display function 162 assigns brightness and color tone for representing region segmentation information to each of regions into which a thrombus region image has been segmented. Here, the region display function 162 uses table information such as a lookup table (LUT) prepared in advance, for example, to assign color information representing each color of red (R), green (G), and blue (B) and information on transparency (α) representing transparency (translucency in some cases) to each pixel constituting the same region. The region display function 162 may display a pattern in the same region. The region display function 162 may make each region have a different transparency such that it is possible to ascertain a state in which different regions overlap. At this time, when there are a plurality of overlapping regions, for example, the region display function 162 may make the transparency of an outer regions higher. The region display function 162 may lower the transparency of a region that the user particularly desires to focus on, such as a region that is expected to have a high fibrin proportion and be hard, to make the region stand out, that is, to improve the visibility of the region that the user particularly wants to focus on. For example, the region display function 162 may increase the transparency of other regions in order to present only a region designated by the user performing an input operation on an input interface (not shown).

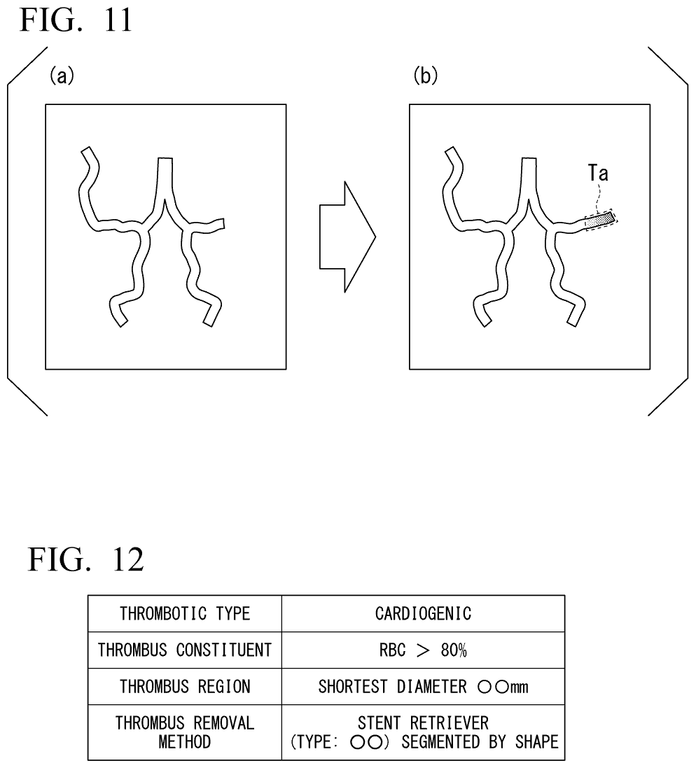

FIG. 11 shows an example of a display image for the region segmentation image processing function 152 to show and display a thrombus region estimated by the thrombus region estimation function 132 on a CT angiography image (CTA image) or MR angiography image (MRA image) output by the medical image acquisition function 122. Here, in cerebral infarction diagnosis, a maximum intensity projection (MIP) image based on a CTA image or an MRA image is often used at the time of visualizing blood vessels without using an angiography image. This is because the movement of blood vessels is easier to ascertain in MIP images. FIG. 11 shows an example of a display image showing a thrombus region in an MIP image. More specifically, (a) of FIG. 11 shows an example of an MIP image generated from a CTA image or an MRA image, and (b) of FIG. 11 shows an example of a display image in which a thrombus region image Ta reflecting the constituent distribution of a thrombus is superimposed on the thrombus region in the MIP image. This allows the user to visually recognize the position of the thrombus relative to blood vessels and the constituent distribution thereof, rather than simply displaying the thrombus region on a CTA image or an MRA image. Accordingly, the user can easily imagine how to advance a thrombus removal device into a blood vessel during surgery to remove a thrombus. For example, if an MIP image is an image configured such that the angle can be changed (e.g., rotated), the region display function 162 may rotate the thrombus region image Ta superimposed on the MIP image depending on the angle of the MIP image.

FIG. 12 shows an example of a display image for the region segmentation image processing function 152 to show estimation results output by the estimation function 130 and a thrombus removal method and a segmentation method output by the determination function 140. FIG. 12 shows an example of a display image showing information on a thrombotic type of “cardiogenic,” information on a thrombus constituent composition of “RBC >80%,” information on a thrombus region of “shortest diameter ○○ mm,” and information on a thrombus removal method and a segmentation method of “stent retriever, (type: ○○) segmented by shape.”

The display function 160 (including the region display function 162) is an example of a “display unit.”

### Processing of Medical Image Processing Device

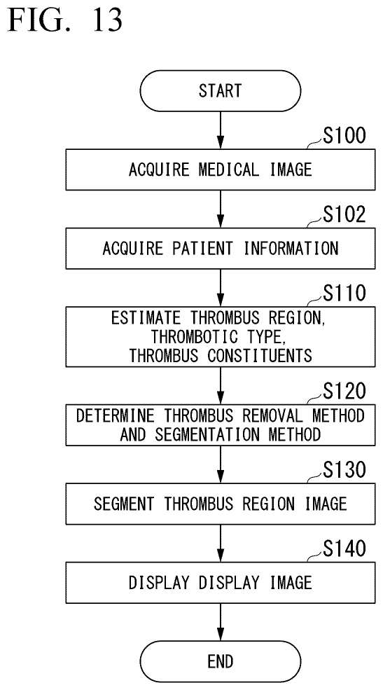

Next, the overall operation of the medical image processing device 100 will be described. FIG. 13 is a flowchart showing an example of a flow of processing in the medical image processing device 100 according to the embodiment.

When processing of presenting information regarding a thrombus is started in the medical image processing device 100, first, the medical image acquisition function 122 included in the acquisition function 120 acquires a medical image (step S100). The medical image acquisition function 122 outputs the acquired medical image to both the estimation function 130 and the display function 160. Further, the patient information acquisition function 124 included in the acquisition function 120 acquires patient information (step S102). The patient information acquisition function 124 outputs the acquired patient information to both the estimation function 130 and the display function 160.

The estimation function 130 estimates a thrombus region, a thrombotic type, and a constituent composition on the basis of the medical image and the patient information output by the acquisition function 120 (step S110). The estimation function 130 outputs an estimation result regarding the estimated thrombus. More specifically, the thrombus region estimation function 132 included in the estimation function 130 estimates a thrombus region that is considered to be a thrombus captured in the medical image, and outputs a thrombus region image as an estimation result to each of the determination function 140, the segmentation function 150, and the display function 160. The thrombotic type estimation function 134 included in the estimation function 130 estimates the thrombotic type of the thrombus that is considered to be captured in the medical image, and outputs thrombotic type information as an estimation result to each of the determination function 140, the segmentation function 150, and the display function 160. The thrombus constituent estimation function 136 included in the estimation function 130 estimates a thrombus constituent composition of the thrombus that is considered to be captured in the medical image, and outputs thrombus constituent composition information as an estimation result to each of the determination function 140, the segmentation function 150, and the display function 160.

The determination function 140 determines a thrombus removal method and a segmentation method on the basis of each of the estimation results output by the estimation function 130 (step S120). The determination function 140 outputs the determined thrombus removal method and segmentation method to the segmentation function 150 and the display function 160 as determination results. More specifically, the determination function 140 determines a thrombus removal method for removing the thrombus captured in the medical image, and outputs the determined thrombus removal method to the segmentation function 150 and the display function 160. The region segmentation determination function 142 determines a segmentation method for the thrombus region captured in the medical image, and outputs the determined segmentation method to the segmentation function 150 and the display function 160.

The segmentation function 150 (including the region segmentation image processing function 152) segments the thrombus region image output by the thrombus region estimation function 132 according to the thrombus removal method and the segmentation method output by the determination function 140 (step S130). The segmentation function 150 (including the region segmentation image processing function 152) outputs region segmentation information representing feature values of regions into which the thrombus region image has been segmented to the display function 160.

The display function 160 (including the region display function 162) generates a display image including information output by each component of the medical image processing device 100, and outputs the generated display image to a display device (not shown) for display (step S140). In this manner, information on the thrombus processed by the medical image processing device 100 is presented to the user.

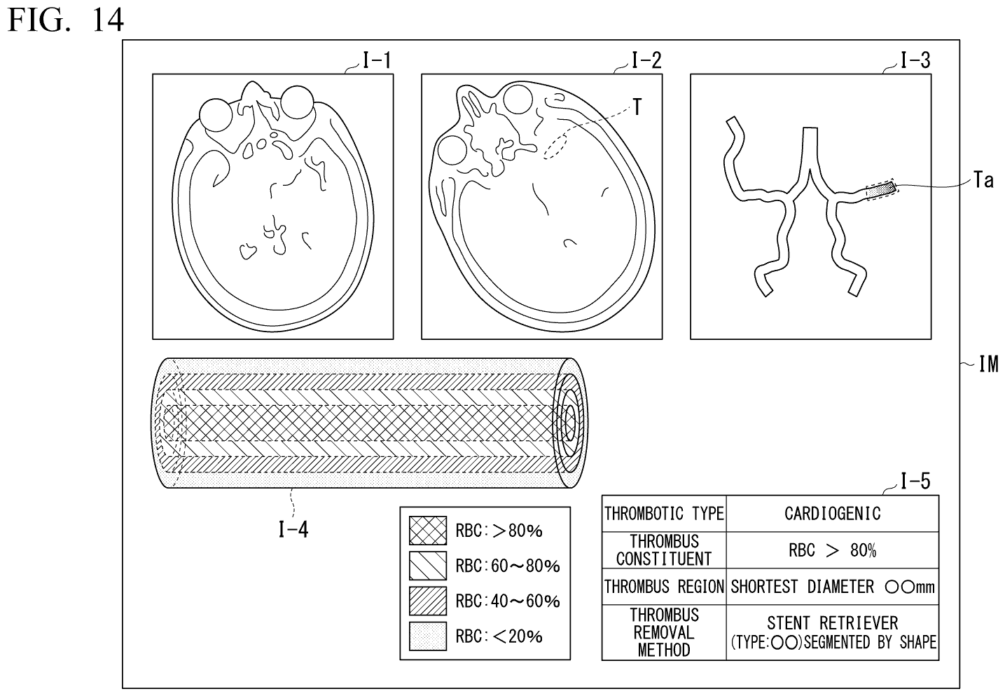

Here, an example of information on the thrombus presented to the user by the medical image processing device 100 will be described. FIG. 14 is a diagram showing an example of a display screen when the medical image processing device 100 according to the embodiment provides information. FIG. 14 shows an example of a display screen IM displayed on a display device (not shown) caused by the medical image processing device 100 to display a single display image that is a combination of a plurality of display images generated by the region display function 162.

In the display screen IM, an NCCT image I-1, a CTA image I-2, an MIP image I-3, a display image I-4, and a display image I-5 are combined and displayed on a display device (not shown). The NCCT image I-1 is an NCCT image acquired by the medical image acquisition function 122. The CTA image I-2 is a CTA image acquired by the medical image acquisition function 122. A thrombus T is captured in the CTA image I-2. The MIP image I-3 is a display image (refer to (b) of FIG. 11) generated by the region segmentation image processing function 152. A thrombus region image Ta is superimposed on the MIP image I-3 by the region segmentation image processing function 152 at a position corresponding to the region of the thrombus T captured in the CTA image I-2. The display image I-4 is a display image (refer to FIG. 10) generated by the region segmentation image processing function 152. The display image I-4 shows the hardness of each of regions (four regions) into which the thrombus T has been segmented. The display image I-5 is a display image (refer to FIG. 12) generated by region segmentation image processing function 152. The display image I-5 shows the state of the thrombus T (thrombotic type, thrombus constituent composition, and thrombus region) and information on a thrombus removal method and a segmentation method corresponding to the thrombus T.

The display screen IM shown in FIG. 14 is merely an example, and a medical image or a display image displayed on the display screen IM may be another medical image or display image instead of or in addition to the medical image or display image displayed on the display screen IM.

In this manner, the medical image processing device 100 presents information on a thrombus that is considered to be captured in a medical image (information more suitable for diagnosing and treating the thrombus) to the user. As a result, the user who uses the medical image processing device 100 can check various types of information on a thrombus that has occurred in a patient, including a constituent distribution (constituent composition of a local region) and removal of the thrombus. This allows the user to make a suitable diagnosis of the thrombus that has occurred in the patient. The user can then make a suitable decision regarding the treatment of the thrombus, such as a surgical procedure for removing the thrombus that has occurred in the patient (selection of a thrombus removal device and a procedure), for example. Accordingly, it is expected to improve the outcome (treatment outcome) of the treatment of the thrombus that has occurred in the patient.

As described above, the medical image processing device of the embodiment acquires a medical image obtained by capturing a patient and patient information. Then, the medical image processing device of the embodiment estimates the state of a thrombus captured in the medical image on the basis of the acquired medical image and patient information. Further, the medical image processing device of the embodiment presents information such as the constituent distribution of the thrombus and the removal of the thrombus to the user on the basis of a result of estimation of the state of the thrombus. This allows the user to make appropriate decision regarding diagnosis and treatment on the basis of information regarding the thrombus presented by the medical image processing device of the embodiment. In other words, the medical image processing device of the embodiment can provide support for diagnosis and treatment of the thrombus performed by the user.

Although an example of a case in which the processing circuitry included in the medical image processing device is realized by a single computer device or a server device on a network (not shown) has been described in the above-described embodiment, this is merely an example, and the processing circuitry included in the medical image processing device, or the functions realized by the processing circuitry included in the medical image processing device may be realized by a configuration in which a plurality of server devices or computer devices are combined. In this case, the functional configuration, operation, and processing of the processing circuitry included in the medical image processing device may be equivalent to the functional configuration, operation, and processing of the processing circuitry included in the medical image processing device in the above-described embodiment. Therefore, the detailed description of the processing circuitry included in the medical image processing device in this case or the functional configuration, operation, and processing realizing the functions thereof will be omitted.

The above-described embodiment can be represented as follows.

A medical image processing device including processing circuitry,
wherein the processing circuitry is configured to:
acquire a medical image in which at least blood vessels of a patient are captured and patient information regarding the patient;
estimate a state of a thrombus captured in the medical image on the basis of the medical image and the patient information, and output an estimation result relating to the estimated thrombus;
determine a thrombus removal method for removing the thrombus on the basis of the medical image, the patient information, and the estimation result, and output a result of determination of the thrombus removal method; and
generate a display image for presenting one or a plurality of the medical image, the estimation result, and the determination result, and cause a display device to display the display image.

According to at least one embodiment described above, it is possible to realize a medical image processing device and a medical image processing method capable of presenting more suitable information for diagnosing and treating a thrombus by including processing circuitry that acquires a medical image in which at least blood vessels of a patient are captured and patient information on the patient (120, 122, and 124), estimates the state of a thrombus captured in the medical image on the basis of the medical image and the patient information, outputs an estimation result regarding the estimated thrombus (130), determines a thrombus removal method for removing the thrombus on the basis of the medical image, the patient information, and the estimation result, outputs a result of determination of the thrombus removal method (140), generates a display image for presenting one or a plurality of the medical image, the estimation result, and the determination result, and causes a display device to display the display image (160 and 162).

Although several embodiments have been described, these embodiments are presented as examples and are not intended to limit the scope of the invention. These embodiments can be implemented in various other forms, and various omissions, substitutions, and modifications can be made without departing from the spirit of the invention. These embodiments and modifications thereof are included in the scope and spirit of the invention, as well as the scope of the invention described in the claims and equivalents thereof.

What is claimed is:

1. A medical image processing device comprising processing circuitry configured to:
acquire a medical image in which at least blood vessels of a patient are captured and patient information regarding the patient;
estimate a state of a thrombus captured in the medical image based on the medical image and the patient information, and output an estimation result relating to the estimated thrombus;
determine a thrombus removal method for removing the thrombus based on the medical image, the patient information, and the estimation result, and output a result of determination of the thrombus removal method; and
generate a display image for presenting any one or a plurality of the medical image, the estimation result, and the determination result and cause a display device to display the display image.

2. The medical image processing device according to claim 1, wherein the processing circuitry estimates a region of the thrombus in the medical image, outputs a thrombus region image of the estimated region of the thrombus as the estimation result, estimates a disease type of the thrombus, outputs thrombotic type information representing the estimated disease type of the thrombus as the estimation result, estimates constituents of the thrombus, and outputs thrombus constituent composition information representing the estimated constituents of the thrombus as the estimation result.

3. The medical image processing device according to claim 2, wherein the processing circuitry outputs the thrombus region image separated from the medical image as the estimation result using a trained model trained to output an image in which a region showing signs of thrombus that is able to be confirmed on the medical image is determined as the region of the thrombus when the medical image is input, outputs the thrombotic type information estimated from the medical image before thrombus removal as the estimation result using a trained model trained to output the disease type of the thrombus identified after thrombus collection when image feature amounts of the medical image before thrombus removal are input, and outputs the thrombus constituent composition information estimated from the medical image before thrombus removal as the estimation result using a trained model trained to output an analysis result after thrombus collection when image feature amounts of the medical image before thrombus removal are input.

4. The medical image processing device according to claim 2, wherein the processing circuitry determines the thrombus removal method based on a combination of the thrombus region image, the thrombotic type information, and the thrombus constituent composition information.

5. The medical image processing device according to claim 4, wherein the processing circuitry determines a segmentation method of segmenting the region of the thrombus represented by the thrombus region image based on the combination of the thrombus region image, the thrombotic type information, and the thrombus constituent composition information, and the thrombus removal method, outputs a result of determination of the segmentation method, performs image processing for segmenting the thrombus region image into a plurality of regions according to the determination result and the result of determination of the segmentation method, and outputs region segmentation information representing feature values including constituents of the thrombus in the segmented regions.

6. The medical image processing device according to claim 5, wherein the thrombus removal method includes information representing the hardness of the thrombus, and the processing circuitry performs the image processing for segmenting the thrombus region image into the regions depending on the hardness of the thrombus.

7. The medical image processing device described in claim 6, wherein the thrombus removal method includes information representing the disease type of the thrombus, and the processing circuitry performs the image processing for segmenting the thrombus region image into the regions depending on the disease type of the thrombus.

8. The medical image processing device according to claim 7, wherein the thrombus removal method includes a procedure for removing the thrombus using a thrombus removal device, and the processing circuitry performs the image processing for segmenting the thrombus region image into the regions according to the thrombus removal device used in the procedure for removing the thrombus when the determination result is a determination to remove the thrombus through the procedure using the thrombus removal device.

9. The medical image processing device according to claim 8, wherein the processing circuitry calculates constituent proportions corresponding to the feature values using a trained model trained to output a constituent composition including proportions of constituents constituting the thrombus represented by the feature values when the feature values are input, and outputs the calculated constituent proportions as the region segmentation information.

10. The medical image processing device according to claim 9, wherein the processing circuitry generates the display image showing the region segmentation information corresponding to each of the segmented regions, and causes the display device to display the display image.

11. A medical image processing method, using a computer, comprising:
acquiring a medical image in which at least blood vessels of a patient are captured and patient information regarding the patient;
estimating a state of a thrombus captured in the medical image based on the medical image and the patient information, and outputting an estimation result relating to the estimated thrombus;
determining a thrombus removal method for removing the thrombus based on the medical image, the patient information, and the estimation result, and outputting a result of determination of the thrombus removal method; and
generating a display image for presenting any one or a plurality of the medical image, the estimation result, and the determination result and causing a display device to display the display image.
